(19) United States
(12) Patent Application Publication
(10) Pub. No.: US 2026/0341409 A1
(43) Pub. Date: Oct. 29, 2026

(54) METHOD FOR IDENTIFYING OIL-DISPLACING MICROORGANISMS IN OIL RESERVOIRS BASED ON METAGENOMICS AND METATRANSCRIPTOMICS

(71) Applicant: EAST CHINA UNIVERSITY OF SCIENCE AND TECHNOLOGY, Shanghai (CN)

(72) Inventors: Bozhong MU, Shanghai (CN); Yifan LIU, Shanghai (CN); Libin SHOU, Shanghai (CN)

(73) Assignee: EAST CHINA UNIVERSITY OF SCIENCE AND TECHNOLOGY, Shanghai (CN)

(21) Appl. No.: 19/115,237

(22) PCT Filed: Jun. 8, 2023
(86) PCT No.: PCT/CN2023/099026

§ 371 Date: Mar. 25, 2025

(30) Foreign Application Priority Data
Sep. 26, 2022 (CN) 202211176719.1

(51) Int. Cl. G16B 30/20; C12Q 1/04; C12Q 1/6806; C12Q 1/6869; G16B 10/00; G16B 20/00; G16B 30/10; G16B 50/00

(52) U.S. Cl. CPC G16B 30/20; C12Q 1/04; C12Q 1/6806; C12Q 1/6869; G16B 10/00; G16B 20/00; G16B 30/10; G16B 50/00

(57) ABSTRACT

The present invention relates to a method for identifying oil-displacing microorganisms in oil reservoirs based on metagenomics and metatranscriptomics, including the following steps: S1: Extracting total DNA and total RNA from the production water of oil reservoirs; S2: Sequencing the obtained total DNA and total RNA to acquire the raw metagenomic and metatranscriptomic data of the oil reservoir samples; S3: Analyzing the metagenomic and metatranscriptomic data to identify oil-displacing microorganisms.

### TECHNICAL FIELD

The present invention relates to the field of microbial detection technology, particularly to a method for identifying oil-displacing functional microorganisms in oil reservoirs based on metagenomics and metatranscriptomics.

### BACKGROUND OF RELATED ART

The heavy oil resources in China are estimated to be approximately 19.87 billion tons, with annual production reaching 30.87 million tons (in 2017), accounting for 16.2% of the total crude oil output. Due to the high viscosity of heavy oil (ranging from 50 to 10,000 mPa's), its poor fluidity within reservoirs necessitates steam-assisted thermal recovery as the primary extraction method. However, thermal recovery methods are energy intensive, costly, and often yield unsatisfactory extraction efficiency. Moreover, oil reservoirs function as natural bioreactors, containing diverse aerobic and anaerobic microorganisms with various functionalities. Microbial enhanced oil recovery (MEOR) is an environmentally friendly and cost-effective technology that can be applied to heavy oil extraction. The primary mechanisms involve microbial emulsification of crude oil, gas production, biosurfactant generation, polysaccharide production, and hydrocarbon degradation. These processes contribute to reducing oil viscosity and enhancing water-flooding efficiency.

Over the past two decades, research on microorganisms in oil reservoirs has evolved from traditional isolation and culture methods to sequencing-based molecular biology technology. Sequencing techniques can be broadly categorized into PCR-dependent and PCR-independent methods (metagenomic and metatranscriptomic sequencing). The former, represented by 16S rRNA gene clone library analysis, has been widely used in studies of oil reservoir samples. Relevant gene sequences from samples can be amplified by designing specific primers, it is allowed to infer the metabolic potential of microbial communities at the genetic level. The latter, which does not rely on PCR amplification, enables simultaneous sequencing of all genes present in a sample. Therefore, metagenomic sequencing analysis of oil reservoir samples provides deeper insights into potential metabolic networks. The integration of metagenomic and metatranscriptomic techniques allows for assessing gene transcription levels within metabolic pathways, making it possible to reveal the microbial metabolic processes in oil reservoirs.

Existing metagenomic analysis methods (e.g., a method for activity quantification and host identification of environmental antibiotic resistance gene based on metatranscriptomics and metagenomics, application No. 202110740585.0) can be used to analyze target functional genes and key microorganisms in conventional environmental samples as needed. However, subsurface oil reservoirs are unique anaerobic environments. The microbial composition of samples is highly susceptible to disruption and RNA is also prone to degradation if sample collection and extraction processes are not specifically optimized, resulting in the inability to obtain in-situ microbial data in subsequent analyses. Moreover, the functional diversity of microorganisms in oil reservoirs is extensive, with many research-worthy species. A single database is insufficient for comprehensive functional analysis, necessitating the integration of multiple public databases along with local databases for a more thorough annotation and analysis of key microbial functions.

Therefore, given the current technological gap, it is urgent to develop a microbial identification and analysis method based on metagenomics and metatranscriptomics specifically tailored for oil reservoir environments.

### SUMMARY OF THE INVENTION

The objective of the present invention is to address the issue in the prior art where the information obtainable through microbial detection methods for oil reservoirs is limited. The present invention provides a method to accurately identify microorganisms and their metabolic functions in oil reservoirs with metagenomics and metatranscriptomics.

The objective of this invention is achieved through the following technical approach:

The objective of this invention is to provide a method for identifying oil-displacing microorganisms in oil reservoirs based on metagenomics and metatranscriptomics, comprising the following steps:
S1: Extracting total DNA and total RNA from water sample produced from oil reservoirs.
S2: Sequencing the obtained total DNA and total RNA to acquire the raw metagenomic and metatranscriptomic of the oil reservoir samples.
S3: Analyzing the results of the metagenomic and metatranscriptomic to identify oil-displacing microorganisms.

Furthermore, for S1: to prevent RNA degradation by anaerobic microorganisms, the inhibitor should be added to the sample before total DNA and total RNA extraction.

Furthermore, for S2: pre-processing the raw metagenomic and metatranscriptomic data of the oil reservoir samples to remove adapters and reads with low quality and get the target data.

The pre-processing procedure includes:

The fastp software is used to perform sliding window quality trimming on the raw paired-end sequence data of each DNA or RNA chain sequence in the metagenomics and metatranscriptomics of the samples of the oil reservoir. Based on the primer information at the first and last ends of the sequence, the primers are removed using a Cutadapt software to obtain the quality-controlled paired-end sequence data.

Furthermore, the parameters of sliding window trimming parameter are set to −W4, −M20, indicating the window size is 4 and the average quality is 20.

Furthermore, for S3: the process of analyzing the results of the metagenomics and metatranscriptomics includes:
Assembling, binning, and quality assessing of the quality-controlled paired-end sequence data, removing the redundancy and extracting the high-quality MAGs (Metagenome-Assembled Genomes) dataset;
Annotating the assembled data of the high-quality MAGs based on a constructed reference databases to identify the functional genes and MAGs with different oil-displacing functions, and calculating the corresponding sequencing depth and relative abundance;
High-quality MAGs dataset are used for phylogenetic analysis to perform community structure analysis of the oil-displacing microorganisms;
Comparing the quality-controlled short-read sequences obtained from metatranscriptomics sequencing after filtering with the dataset of the MAGs to calculate the transcriptional levels of each genes.

Furthermore, for S3: the process of assembling, binning, and quality assessing includes:
Assembly: Assembling the quality-controlled paired-end sequence data into contigs with different lengths by SPADes Assembler under Meta mode, connecting different contigs (contiguous sequences) into scaffolds (genomic scaffolds) with sequence gaps based on the paired-end information;
Binning: Sequencing coverage of different contig sequences can be obtained by mapping the quality-controlled short-read MAGs to contig sequences with the bowtie2 software. Further, separating the genomes of dominant microorganisms from the MAGs through three binning methods of Maxbin2, Metabat2, and CONCOCT, which are then imported into the DAS_Tool program for evaluation, ultimately integrating and improving the quality of genomes generated by different methods;
Quality assessment: Removing redundant sequences from the genomes with high similarity from the quality-controlled MAGs using dRep software, and estimating genome completeness and contamination with a CheckM tool according to the presence and quantity of single-copy marker genes in the genomes.

Furthermore, for S3: the process of annotating the high-quality MAGs assembly data includes:
Translating the assembled contig sequences into coding protein sequence (CDS) with Prodigal program (Open reading frame), which are then submitted to the KEGG database for functional annotation using the GhostKOALA tool to obtain KO numbers representing different paralogous subunits;
At the same time, using a local software KofamKOALA to annotate KO numbers for each protein sequences based on the hidden Markov models and recommended confidence thresholds of each KO paralogous family proteins;
Finally, using EggNOG emapper 2 to annotate COG numbers for protein sequences, which are then converted into KO numbers, such that the final KO number annotation for each protein follows the order: 1) GhostKOALA KO, 2) KofamKOALA KO, 3) EggNOG emapper KO.

Furthermore, for S3, the oil-displacing functions include one or more of the following: alkane degradation, gas production, emulsifier production, surfactant production, and polysaccharide production.

Furthermore, for S3, the process of identifying functional genes and MAGs with different oil-displacing functions includes:
For hydrogenase, firstly identifying potential functional gene proteins based on the local HMM Model alignment of hydrogenase subgroups;
Then submitting the potential functional gene protein sequences to the online analysis software of HydDB database to further classify hydrogenase subtypes;
For potential functions of encoding secondary metabolite in the genome, submitting the genome sequences to the AntiSMASH website and combining various tools to identify potential genes encoding secondary metabolite and predict the type of metabolites;
For functional genes with missing information in the database, construct a local protein sequence database. Using the BlastP (Blast Protein) alignment to find the most similar protein sequences for further analysis. The functional genes with missing information in the database include one or more of the following: genes involved in the anaerobic hydrocarbon degradation AssA, EbdA, AhyA, AncA and bacterial microcompartment protein cluster genes.

Furthermore, for S3, the process of phylogenetic analysis on the high-quality MAGs dataset includes:

Firstly, downloading and merging the target sequences with similar reference sequences from the database, firstly aligning the merged sequences with MAFFT, selecting conserved sites with an 80% threshold. Using IQ-tree (The algorithm uses the maximum likelihood method algorithm to construct phylogenetic tree) for comparing sequences to generate a maximum likelihood phylogenetic tree.

Furthermore, for S3, the process of comparing the quality-controlled short-read sequences obtained from metatranscriptomics sequencing after filtering with the MAGs dataset to calculate the transcriptional levels of genes includes:

Mapping the short-read cDNA from the high-quality MAGs dataset to the contig sequences assembled from the metagenome with bowtie2 software. Calculating the transcriptional levels of genes, expressed as TPM (Transcripts Per Million) values.

Compared to existing technologies, the advantages of this method include:

This method is a microbial analysis technology based on metagenomics and metatranscriptomics, which is specifically designed for samples from oil reservoirs. In the whole experiment process, extract total DNA and total RNA from the water sample produced from oil reservoirs and perform sequencing to acquire the raw metagenomics and metatranscriptomics data of the oil reservoir samples, analyze the metagenomic and metatranscriptomic data to identify oil-displacing microorganisms. The whole experiment process does not rely on those traditional, time-consuming methods of microbial isolation and identification, making it suitable for the detection of samples with a high proportion of unknown species. This invention allows for comprehensive and rapid detection, including species with extremely low abundance.

### DETAILED DESCRIPTION OF THE EMBODIMENTS

The following describes the invention in detail through specific embodiments, which are not the limitation of the invention. Any software/program names, control methods, algorithms, or other features not explicitly specified in this technical solution are considered as common technical features disclosed in the prior art.

The method for identifying oil-displacing microorganisms in oil reservoirs based on metagenomic and metatranscriptomic sequencing, which is proposed in this invention specifically includes the following steps:
Step s1: Collect environmental samples and add RNALater reagent to inhibit RNA degradation. Then extract DNA and RNA separately using DNA and RNA extraction kits.
Step s2: Perform metagenomics and metatranscriptomics sequencing.
Step s3: Analyze the metagenomics and metatranscriptomics sequencing data, including the following steps:
Step s31: Use fastp to perform quality control on the short-read sequences, and remove the low-quality sequences and adaptors.
Step s32: Assemble the quality-controlled short sequences into ‘contigs’ with different lengths by SPADes Assembler in Meta mode. Connect different ‘contigs’ into ‘scaffolds’ with gaps based on the paired-end information.
Step s33: Translate the assembled contig sequences into coding protein sequence (CDS) with Prodigal program. Annotate COG numbers for protein sequences with three tools of GhostKOALA, KofamKOALA, EggNOG emapper 2, which are then converted into KO numbers. The final KO number annotation for each protein follows the order: 1) GhostKOALA KO, 2) KofamKOALA KO, 3) EggNOG-mapper KO.
Step s34: For hydrogenase, identify potential functional gene proteins based on the local HMM Model alignment of hydrogenase subgroups. Then submitting the potential functional gene protein sequences to the online analysis platform of HydDB database to further classify hydrogenase subtypes. For potential functions of encoding secondary metabolite in the genome, submitting the genome sequences to the AntiSMASH website and using various tools to identify potential genes encoding secondary metabolite and predict the type of metabolites. Moreover, for functional genes with missing information in the database, genes involved in the anaerobic hydrocarbon degradation AssA, EbdA, AhyA, AncA and bacterial microcompartment protein cluster genes, constructing a local protein sequence database. Using the BlasP alignment to find the most similar protein sequences for further analysis.
Step s35: Separate the genomes from the MAGs through three binning methods of Maxbin2, Metabat2, CONCOCT, which are then imported into the DAS_Tool program for evaluation, ultimately integrating and improving the quality of genomes generated by different methods.
Step s36: Downloading and merging the target sequences with similar reference sequences from the database, aligning the merged sequences using MAFFT, selecting conserved sites with an 80% threshold. Using IQ-tree for comparing sequences to generate a maximum likelihood phylogenetic tree.
Step s37: for short-read mRNA sequence obtained by metatranscriptome sequencing, map the short-read cDNA to the contig DNA sequences assembled from the metagenome with bowtie2 software. Calculate the transcriptional levels of genes, expressed as TPM (Transcripts Per Million) values.

### Embodiments 1

### (1) Sample Collection and Nucleic Acid Extraction

RNALater was previously added to the sample container at 20% of the total volume to prevent RNA degradation. The production water sample from oil reservoirs was ensured to completely fill the container to eliminate air. The samples were stored at low temperatures before nucleic acid extraction.

DNA and RNA were extracted using the PowerSoil DNA Kit (QIAGEN, USA) and PowerSoil RNA Kit (QIAGEN, USA), respectively. The extracted RNA was further purified using an RNase-Free DNase Set (QIAGEN, USA), and complementary DNA (cDNA) was synthesized and stored at −80 °C.

### (2) Microbial Metagenomic and Metatranscriptomic Data

A sequencing library with index codes was constructed using the NEB Next® Ultra™ DNA Library Prep Kit for Illumina® (New England Biolabs, USA). The quality of the sequencing library was assessed using Qubit 3.0 Fluorometer (Life Technologies, USA) and Agilent 4200 system (Agilent, Canada). Finally, sequencing was performed on the Illumina HiSeq X-ten platform, generating 150 bp paired-end nucleotide sequences.

### (3) Data Analysis

The raw sequencing data were subjected to quality control and filter using fastp. Metagenomic data were assembled using SPAdes and binned using Maxbin2, Metabat2, and CONCOCT. The genomes were further imported into the DAS_Tool to obtain high-quality genomes. The assembled contig sequences are translated into coding protein sequence (CDS) using Prodigal program. Protein sequences are annotated with three tools, which are GhostKOALA, KofamKOALA and EggNOG emapper 2. The potential of hydrogen production and secondary metabolite biosynthesis was analyzed using the HydDB database and AntiSMASH. The potential of anaerobic hydrocarbon degradation was assessed by constructing a local sequence database.

Metatranscriptomics data were aligned to the assembled contig DNA sequences with Bowtie2, and transcription levels for each gene were calculated as TPM.

The dominant microorganisms and their metabolic functions in oil reservoirs could be accurately identified by analyzing the relative abundance of genomes and corresponding metabolic pathways. The expression activity of these microorganisms could be further determined with transcriptomic data.

Take a production water sample from the Daqing Oilfield as an example, the sequencing coverage and metabolic pathways for different genomes were summarized in Table 1. The results revealed the dominant microorganisms in the sample included Kerstersia gyiorum and Serratia nematodiphila of the phylum of Proteobacteria), as well as Enterococcus of the phylum of Firmicutes. Most microorganisms in the sample were nitrate-reducing facultative anaerobes, and aerobic hydrocarbon-degrading bacteria were present for hydrocarbons with varying chain lengths. Nearly half of the microorganisms had complete metabolic pathways for lipopeptide biosynthesis. Most hydrogenotrophic microorganisms had the potential for hydrogen production. These findings provide theoretical insights for enhanced oil recovery and corrosion prevention in oil reservoirs.

TABLE 1
Genome information

| | | | | | Aerobic | |
|---|---|---|---|---|---|---|
| | | | | Aerobic | long-chain | |
| | Sequencing | Acetic acid | | hydrocarbon | hydrocarbon | Fatty acid |
| Genome | coverage | metabolism | Aerobic | degradation | degradation | degradation |
| Alkaliphilus_oremlandii_bin_24 | 0.82 | | | | | |
| Aquabacterium_bin_26 | 3.99 | ✓ | ✓ | ✓ | | ✓ |
| Bacillus_cereus_AE_bin_2 | 3.36 | ✓ | ✓ | | | |
| Bacillus_licheniformis_bin_11 | 0.96 | ✓ | ✓ | | | |
| Bacillus_paralicheniformis_bin_5 | 0.22 | ✓ | ✓ | | | |
| Bacillus_thermoamylovorans_bin_1 | 0.19 | ✓ | ✓ | | | ✓ |
| Brevibacillus_borstelensis_bin_18 | 0.88 | ✓ | ✓ | | | |
| Brevibacillus_borstelensis_bin_6 | 0.19 | ✓ | | | | |
| Burkholderiaceae_bin_19 | 7.84 | ✓ | ✓ | | | ✓ |
| Burkholderiaceae_bin_5 | 15.83 | ✓ | ✓ | | | ✓ |
| Clostridium_G_cochlearium_bin_14 | 21.26 | | | | | |
| Clostridium_J_argentinense_bin_24 | 0.73 | | | | | |
| Enterococcus_faecium_bin_1 | 45.73 | | | | | |
| Enterococcus_faecium_bin_8 | 323.94 | ✓ | | | | |
| Escherichia_coli_bin_13 | 8.71 | ✓ | ✓ | | | ✓ |
| Escherichia_coli_bin_15 | 0.19 | ✓ | ✓ | | | ✓ |
| Gemmobacter_nectariphilus_bin_2 | 6.78 | ✓ | ✓ | ✓ | ✓ | ✓ |
| Kerstersia_gyiorumbin_2 | 1772.74 | ✓ | ✓ | | ✓ | ✓ |
| Leucobacter_bin_9 | 8.89 | ✓ | ✓ | | ✓ | ✓ |
| Pseudomonas_formosensis_bin_23 | 0.37 | ✓ | ✓ | | | ✓ |
| Pseudomonas_formosensis_bin_24 | 0.24 | ✓ | ✓ | | ✓ | ✓ |
| Pseudomonas_thermotolerans_bin_8 | 0.46 | ✓ | ✓ | ✓ | ✓ | ✓ |
| Roseovarius_bin_1 | 3.56 | ✓ | ✓ | ✓ | | |
| Serratia_nematodiphila_bin_12 | 193.18 | ✓ | ✓ | | ✓ | ✓ |
| Thauera_bin_4 | 41.11 | ✓ | ✓ | | | ✓ |
| Thauera_phenylacetica_bin_19 | 6.45 | ✓ | ✓ | | | ✓ |
| Tissierellaceae_bin_14 | 0.47 | | | | | |

| | Carbohydrate | Nitrate | | Hydrogen | Ethanol | Lipopeptide |
|---|---|---|---|---|---|---|
| Genome | degradation | reduction | Hydrogenotrophic | production | fermentation | synthesis |
| Alkaliphilus_oremlandii_bin_24 | ✓ | ✓ | | | ✓ | |
| Aquabacterium_bin_26 | ✓ | ✓ | | | ✓ | |
| Bacillus_cereus_AE_bin_2 | ✓ | | | | ✓ | |
| Bacillus_licheniformis_bin_11 | ✓ | ✓ | | | ✓ | ✓ |
| Bacillus_paralicheniformis_bin_5 | | ✓ | | | ✓ | ✓ |
| Bacillus_thermoamylovorans_bin_1 | ✓ | ✓ | | | ✓ | |
| Brevibacillus_borstelensis_bin_18 | ✓ | | | | ✓ | ✓ |
| Brevibacillus_borstelensis_bin_6 | | | | | ✓ | ✓ |
| Burkholderiaceae_bin_19 | ✓ | ✓ | | | ✓ | |
| Burkholderiaceae_bin_5 | ✓ | ✓ | | | ✓ | |
| Clostridium_G_cochlearium_bin_14 | ✓ | ✓ | ✓ | | ✓ | |
| Clostridium_J_argentinense_bin_24 | ✓ | ✓ | ✓ | | ✓ | ✓ |
| Enterococcus_faecium_bin_1 | | | | | | |
| Enterococcus_faecium_bin_8 | ✓ | | | | ✓ | |
| Escherichia_coli_bin_13 | ✓ | ✓ | ✓ | ✓ | ✓ | ✓ |
| Escherichia_coli_bin_15 | | ✓ | ✓ | ✓ | ✓ | |
| Gemmobacter_nectariphilus_bin_2 | ✓ | | | | ✓ | |
| Kerstersia_gyiorumbin_2 | ✓ | | | | ✓ | |
| Leucobacter_bin_9 | ✓ | ✓ | | | ✓ | ✓ |
| Pseudomonas_formosensis_bin_23 | ✓ | ✓ | | | ✓ | |
| Pseudomonas_formosensis_bin_24 | ✓ | ✓ | | | ✓ | |
| Pseudomonas_thermotolerans_bin_8 | | ✓ | | | ✓ | |
| Roseovarius_bin_1 | ✓ | ✓ | | | ✓ | |
| Serratia_nematodiphila_bin_12 | ✓ | ✓ | | | ✓ | ✓ |
| Thauera_bin_4 | ✓ | ✓ | ✓ | ✓ | ✓ | |
| Thauera_phenylacetica_bin_19 | ✓ | ✓ | ✓ | ✓ | ✓ | ✓ |
| Tissierellaceae_bin_14 | ✓ | ✓ | | | ✓ | |

The description of the embodiments is provided to facilitate understanding and application of the invention by those skilled in the art. It is evident that individuals familiar with the field can readily make various modifications to these embodiments and apply the general principles described herein to other embodiments without requiring inventive effort. Therefore, the present invention is not limited to the embodiments described above. Any improvements and modifications made by those skilled in the art based on the disclosures of this invention, without departing from its scope, should be considered within the protection scope of the present invention.

1. A method for identifying oil-displacing microorganisms in oil reservoirs based on metagenomics and metatranscriptomics, comprising the following steps:
S1: extracting total DNA and total RNA from water sample produced from oil reservoirs;
S2: performing sequencing to the obtained total DNA and the total RNA to acquire raw data of metagenomics and metatranscriptomics of sample of the oil reservoirs; and
S3: identifying oil-displacing microorganisms by analyzing results of the metagenomics and metatranscriptomics.

2. The method for identifying oil-displacing microorganisms in oil reservoirs based on metagenomics and metatranscriptomics according to claim 1, wherein in S1, before extracting the total DNA and the total RNA, adding an inhibitor to the sample to be extracted RNA to inhibit RNA degradation by anaerobic microorganisms.

3. The method for identifying oil-displacing microorganisms in oil reservoirs based on metagenomics and metatranscriptomics according to claim 1, wherein in S2, further comprising: pre-processing the raw data of the metagenomics and metatranscriptomics of the sample of the oil reservoir to obtain target data with adapters and low-quality reads removed;
wherein a process of the pre-processing includes:
using a fastp software to perform sliding window quality trimming on raw paired-end sequence data of each DNA or RNA chain sequence in the metagenomics and metatranscriptomics of the sample of the oil reservoir, and at the same time, based on primer information at both ends of the sequence, removing primers by using Cutadapt software, and obtaining the quality-controlled paired-end sequence data.

4. The method for identifying oil-displacing microorganisms in oil reservoirs based on metagenomics and metatranscriptomics according to claim 3, wherein in S3, a process of the analyzing the results of the metagenomics and metatranscriptomics includes:
assembling, binning, and evaluating quality of the quality-controlled paired-end sequence data, and extracting a dataset of high-quality Metagenome Assembled Genomes (MAGs) after removing redundancy;
annotating an assembled data of the high-quality MAGs based on a constructed reference databases and identifying functional genes and MAGs with different oil-displacing functions, and calculating corresponding sequencing depth and relative abundance;
performing phylogenetic analysis on the dataset of the high-quality MAGs, and performing community structure analysis of the oil-displacing microorganisms; and
comparing quality-controlled short-read sequences obtained from metatranscriptomics sequencing after filtering with the dataset of the MAGs to calculate transcriptional levels of each genes.

5. The method for identifying oil-displacing microorganisms in oil reservoirs based on metagenomics and metatranscriptomics according to claim 4, wherein in S3, a process of the assembling, binning, and evaluating quality includes:
assembly: assembling the quality-controlled paired-end sequence data into contigs with different lengths by using a SPADes Assembler under Meta mode, connecting the different contigs into scaffolds with sequence gaps based on information of the paired-end sequence data;
binning: mapping the quality-controlled short-read sequences of the MAGs to a contig sequences by using a bowtie2 software to obtain a sequencing coverage information of different contig sequences, further separating genomes of dominant microorganisms from the MAGs by using three binning methods of Maxbin2, Metabat2, and CONCOCT, and further importing into a DAS_Tool program for evaluation, ultimately integrating and improving a quality of the genomes generated by different methods; and
quality assessment: removing redundant sequences from the genomes with high similarity from the quality-controlled MAGs using a dRep software, and estimating completeness and contamination of the genomes through a CheckM tool based on presence and number of single-copy marker genes in the genomes.

6. The method for identifying oil-displacing microorganisms in oil reservoirs based on metagenomics and metatranscriptomics according to claim 4, wherein in S3, a process of the annotating the assembled data of the high-quality MAGs includes:
translating the assembled contig sequences into a coding DNA sequence (CDS) with a Prodigal program, submitting to a KEGG database, and performing functional annotation using a GhostKOALA tool to obtain KEGG Orthology (KO) numbers representing different paralogous subunits;
using a local software KofamKOALA to annotate the KO numbers of each protein sequences based on a hidden Markov model and recommended confidence thresholds of each KO paralogous family proteins; and
using an EggNOG emapper 2 to annotate Clusters of Orthologous Groups (COG) numbers for the protein sequences, which are then converted into the KO numbers, such that the final KO number annotated for each protein follows the order of: 1) GhostKOALA KO, 2) KofamKOALA KO, 3) EggNOG emapper KO.

7. The method for identifying oil-displacing microorganisms in oil reservoirs based on metagenomics and metatranscriptomics according to claim 4, wherein in S3, the oil-displacing functions include one or more of the following: alkane degradation, gas production, emulsifier production, surfactant production, and polysaccharide production.

8. The method for identifying oil-displacing microorganisms in oil reservoirs based on metagenomics and metatranscriptomics according to claim 7, wherein in S3, a process of the identifying functional genes and MAGs with different oil-displacing functions includes:
for hydrogenase, identifying potential functional gene proteins based on a local HMM (Hidden Markov Model) alignment of hydrogenase subgroups; and
submitting sequences of the potential functional gene proteins to an online analysis software of HydDB database to further classify hydrogenase subtypes;
for potential functions of encoding secondary metabolite in the genomes, submitting sequences of the genomes to AntiSMASH website, combining various tools to identify potential genes encoding secondary metabolite, and predicting a type of the metabolites; and
for functional genes with missing information in the database, constructing a local protein sequence database, using a BlastP alignment to find the most similar protein sequences for further analysis, the functional genes with missing information in the database include one or more of the following: genes involved in anaerobic hydrocarbon degradation and bacterial microcompartment protein cluster genes,
wherein the genes involved in anaerobic hydrocarbon degradation include AssA, EbdA, AhyA, and AncA.

9. The method for identifying oil-displacing microorganisms in oil reservoirs based on metagenomics and metatranscriptomics according to claim 4, wherein in S3, a process of the phylogenetic analysis on the dataset of the high-quality MAGs includes:
downloading and merging target sequences with similar reference sequences from the database, aligning the merged sequences on an MAFFT software, and selecting conserved sites with an 80% threshold, and followed by using an IQ-tree for comparing sequences pairwise to generate a maximum likelihood phylogenetic tree.

10. The method for identifying oil-displacing microorganisms in oil reservoirs based on metagenomics and metatranscriptomics according to claim 4, wherein in S3, a process of comparing the quality-controlled short-read sequences obtained from the metatranscriptomics sequencing after filtering with the dataset of the MAGs to calculate the transcriptional levels of each genes includes:
mapping short-read cDNA from the dataset of the high-quality MAGs to contig sequences assembled from the metagenomics with the bowtie2 software, and calculating the transcriptional levels of each genes, expressed as Transcripts Per Million (TPM) values.
